(19) United States
(12) Patent Application Publication
(10) Pub. No.: US 2024/0237924 A1
(43) Pub. Date: Jul. 18, 2024

(54) METHOD AND APPARATUS FOR IDENTIFYING A POSTURE CONDITION OF A PERSON

(71) Applicant: NEC Corporation, Tokyo (JP)

(72) Inventors: Ni Ni SOE, Singapore (SG); Charles Chi Hin CHOY, Singapore (SG)

(73) Assignee: NEC Corporation, Tokyo (JP)

(21) Appl. No.: 18/408,710

(22) Filed: Jan. 10, 2024

(30) Foreign Application Priority Data
Jan. 18, 2023 (SG) 10202300152Q

(51) Int. Cl. A61B 5/11; G16H 10/60; G16H 20/30; G16H 50/30

(52) U.S. Cl. CPC A61B 5/1121; A61B 5/1128; G16H 10/60; G16H 20/30; G16H 50/30

(57) ABSTRACT

The present disclosure provides a method and an apparatus for identifying a posture condition of a person, the method comprising: detecting a rotation of one or more body parts of the person around an upright center axis of the person; and calculating an asymmetric score of the one or more body parts based on the rotation of the one or more body parts of the person, the asymmetric score relating to a level of the posture condition of the person.

### INCORPORATION BY REFERENCE

This application is based upon and claims the benefit of priority from Singapore patent application No. 10202300152Q, filed on Jan. 18, 2023, the disclosure of which is incorporated herein in its entirety by reference.

### TECHNICAL FIELD

The present invention relates broadly, but not exclusively, to a method and an apparatus for identifying a posture condition of a person.

### BACKGROUND ART

Posture relates to the relative alignment of various body segments with one another. A good posture means that the body's alignment is balanced so that stress applied to the body segments is minimal, while poor posture means that the body's alignment is out of balance, causing unusual stresses to various body segments, which can lead to abnormal anatomical adaptations, alterations in performance, and less efficiency.

Postural analysis is an assessment of the function of the motor system (bones, muscles, and ligaments) and the nervous system's control of the motor system. More than just a bone and muscle assessment, it also covers spinal cord alignment. With postural analysis, it is possible to investigate correct standing alignment of a person from an anterior view, posterior view and lateral view of the person.

In postural analysis, experienced and skillful physiotherapists can assess a patient's posture with more accuracy and confidence, while junior physiotherapists might struggle to correctly and confidently assess the patient's posture. To date, there is some limitation in assessing posture such as manual assessment by identifying landmark on images, by placing reflected markers on a specific body position and utilizing a pressure mat under the foot of a patient. Most of the calculated deviation from images are with respect to shifting (e.g., left/right) from a reference and tilting (e.g., up/down) of the body main joint, but do not take into account the rotation of body joints.

Further, landmarks (e.g., anatomical points) of body, face and spinal cord are important to assess the overall posture deviation. Especially, spinal cord landmarks are manually provided by clinicians. Posture of each individual is different and it typically starts forming from a younger age of the individual. Besides, deformation of bone structure can also cause deformation of posture in the form of shifting, tilting and rotation. It is thus important to assess the individual posture not only based on identification of any shifting and/or tilting, but also based on identification of any rotation of a body part for all age groups, from pediatric to elderly and from healthy individuals to patients to get early intervention to correct the individual posture.

Herein disclosed are embodiments of a method and apparatus for identifying a posture condition of a person that addresses one or more of the above problems.

Furthermore, other desirable features and characteristics will become apparent from the subsequent detailed description and the appended claims, taken in conjunction with the accompanying drawings and this background of the disclosure.

### SUMMARY

In a first aspect, the present disclosure provides a method for identifying a posture condition of a person, comprising: detecting a rotation of one or more body parts of the person around an upright center axis of the person; and calculating an asymmetric score of the one or more body parts based on the rotation of the one or more body parts of the person, the asymmetric score relating to a level of the posture condition of the person.

In a second aspect, the present disclosure provides an apparatus for identifying a posture condition of a person, comprising: at least one processor; and at least one memory including computer program code; the at least one memory and the computer program code configured to, with at least one processor, cause the server at least to: detect a rotation of one or more body parts of the person around an upright center axis of the person; and calculate an asymmetric score of the one or more body parts based on the rotation of the one or more body parts of the person, the asymmetric score relating to a level of the posture condition of the person

In a third aspect, the present disclosure provides a system for identifying a posture condition of a person comprising the apparatus of the above aspect and one or more image capturing apparatuses configured to capture one or more images of the person, wherein the one or more images comprises an image of the person across a frontal plane and/or an image of the person across a sagittal plane.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

The accompanying Figures, where like reference numerals refer to identical or functionally similar elements throughout the separate views and which together with the detailed description below are incorporated in and form part of the specification, serve to illustrate various embodiments and to explain various principles and advantages in accordance with a present embodiment, by way of non-limiting example only.

Embodiments of the invention will be better understood and readily apparent to one of ordinary skill in the art from the following written description, by way of example only, and in conjunction with the drawings, in which:

FIG. 1 shows an example of assessing the shifting, tilting and the rotation from a human image.

FIG. 2 shows an illustration for imbalance weight bearing due to rotation of a hip of a person.

FIG. 3 shows a flow chart illustrating a method for identifying a posture condition of a person according to various embodiments of the present disclosure.

FIG. 4 shows a block diagram of an apparatus for identifying a posture condition of a person according to an embodiment of the present disclosure.

FIG. 5 shows an overall system workflow diagram for identifying a posture condition of a person according to an embodiment of the present disclosure.

FIG. 6 shows a flowchart for identifying a posture condition of a person according to an embodiment of the present disclosure.

FIG. 7 shows a flowchart for a landmarks detection system according to an embodiment of the present disclosure.

FIG. 8 shows an exemplary illustration for landmarks detection according to an embodiment of the present disclosure.

FIG. 9 shows another exemplary illustration for landmarks detection according to an embodiment of the present disclosure.

FIG. 10 shows an illustration for detection of jugular notch (JN) from a front view according to an embodiment of the present disclosure.

FIG. 11 shows an illustration for detection of jugular notch (JN) from a side view according to an embodiment of the present disclosure.

FIG. 12 shows an exemplary front-view illustration for detection of jugular notch (JN) according to an embodiment of the present disclosure.

FIG. 13 shows an exemplary side-view illustrations for detection of jugular notch (JN) according to an embodiment of the present disclosure.

FIG. 14 shows a frontal view for identification of a central ray (CR) position based on a detected jugular notch (JN) according to an embodiment of the present disclosure.

FIGS. 15A-15B show respectively a right lateral view and left lateral view for detection of central ray (CR) position, chest and mid-chest (e.g., T7) according to an embodiment of the present disclosure.

FIG. 16 shows a right lateral view for estimation of cervical (C7), thoracic (T2, T7, T10), lumbar (L1 and L4) and Sacral (S2) vertebrae position of spinal cord according to an embodiment of the present disclosure.

FIG. 17 shows a left lateral view for estimation of cervical (C7), thoracic (T2, T7, T10), lumbar (L1 and L4) and Sacral (S2) vertebrae position of spinal cord according to an embodiment of the present disclosure.

FIG. 18 shows an anterior/frontal view with detected whole body landmarks according to an embodiment of the present disclosure.

FIGS. 19A-19B show a left lateral view and right lateral view with detected whole body landmarks according to an embodiment of the present disclosure.

FIG. 20 shows a flow chart illustrating for a posture analyzer according to an embodiment of the present disclosure.

FIG. 21 shows a table for calculation of deviation and kinematic parameters for anterior view of body images according to an embodiment of the present disclosure.

FIGS. 22A and 22B show an analysis of position and angle respectively of various body part positions according to an embodiment of the present disclosure.

FIGS. 23A and 23B show an analysis of knee alignment and foot alignment respectively according to an embodiment of the present disclosure.

FIG. 24 shows an illustration for determining body part rotation according to an embodiment of the present disclosure.

FIG. 25 shows a table for calculation of deviation and kinematic parameters for lateral view of body images according to an embodiment of the present disclosure.

FIG. 26A shows an illustration for determining joint shifting from a side view according to an embodiment of the present disclosure.

FIG. 26B shows an illustration for determining joint angle from a side view according to an embodiment of the present disclosure.

FIG. 26C shows an illustration for determining hip and knee angle from a side view according to an embodiment of the present disclosure.

FIG. 27A shows an exemplary illustration of a “forward head/computer neck” posture according to an embodiment of the present disclosure.

FIG. 27B shows an exemplary illustration of a jaw-dropped posture according to an embodiment of the present disclosure.

FIGS. 28A and 28B show exemplary illustrations of posture analyzer output based on a front body view according to an embodiment of the present disclosure.

FIGS. 29A and 29B show exemplary illustrations of posture analyzer output based on a right side and left side body view respectively according to an embodiment of the present disclosure.

FIG. 30 shows a table for calculation of asymmetric scores to provide a level of posture abnormalities according to an embodiment of the present disclosure.

FIG. 31 shows a flow chart illustrating for a posture correction exercise recommender according to an embodiment of the present disclosure.

FIG. 32 shows a schematic diagram of an exemplary computing device suitable for use to execute the method in FIG. 3.

A posture condition of a person relates to how a body part (e.g., shoulder, chin, hip, head, ear, eye brow, ankle, and other similar body parts) of the person deviates from a standard position (e.g., a reference position) of the body part. It can relate to a shift (e.g., left or right shift), tilt (e.g., upward or downward tilt), or rotation from the reference position. The identification may utilize an image of an anterior view, a frontal view and/or a lateral view (e.g., an image of the person across a sagittal plane, such as a right and/or left lateral view) of a body of a person. Based on the image, each body part position may be identified and denoted by a respective landmark (e.g., anatomical point), and any shift, tilt or rotation may be detected based on comparison of lines connecting and/or distances measured between the body part positions, as well as detection and calculation of angles derived from the comparison of lines. For example, a rotation of one or more body parts of the person around an upright center axis of the person may be determined based on detecting an angle of a line connecting two body part positions against a reference line. The reference line may be another line connecting another two body part positions of the person. Different landmarks, lines and/or angles may be utilized depending on the body part of which a posture condition is to be determined.

An asymmetric score relating to a level of a posture condition of a person may be calculated based on a detected rotation of one or more body parts of the person. For example, deviation and kinematic parameters may be calculated based on the identified landmarks, lines and/or angles to obtain the score. For example, a higher asymmetric score indicates a higher severity of deviation for a body part of the person, such that higher emphasis may be placed on correction of the posture of the body part in comparison to other body parts with lower asymmetric scores.

Embodiments of the present invention will be described, by way of example only, with reference to the drawings. Like reference numerals and characters in the drawings refer to like elements or equivalents.

Some portions of the description which follows are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “detecting”, “estimating”, “comparing”, “receiving”, “calculating”, “determining”, “updating”, “generating”, “initializing”, “outputting”, “receiving”, “retrieving”, “identifying”, “dispersing”, “authenticating” or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a computer or other device selectively activated or reconfigured by a computer program stored in the computer. The algorithms and displays presented herein are not inherently related to any particular computer or other apparatus. Various machines may be used with programs in accordance with the teachings herein. Alternatively, the construction of more specialized apparatus to perform the required method steps may be appropriate. The structure of a computer will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a computer. The computer readable medium may also include a hard-wired medium such as exemplified in the Internet system, or wireless medium such as exemplified in the GSM mobile telephone system. The computer program when loaded and executed on such a computer effectively results in an apparatus that implements the steps of the preferred method.

### EXAMPLE EMBODIMENT

Various embodiments of the present disclosure relate to a method and an apparatus for identifying a posture condition of a person.

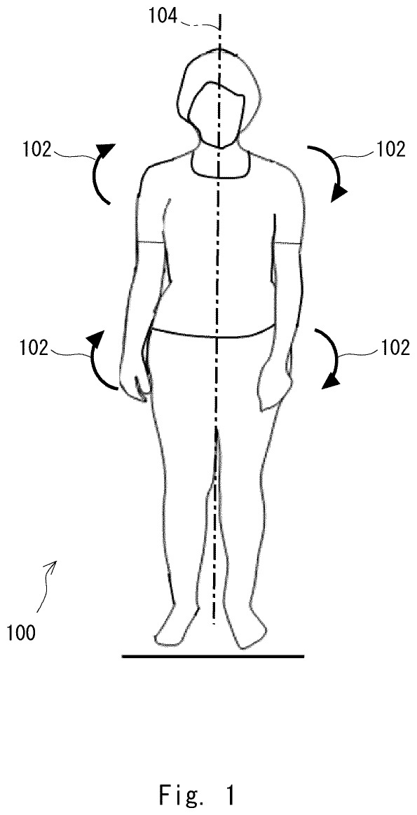

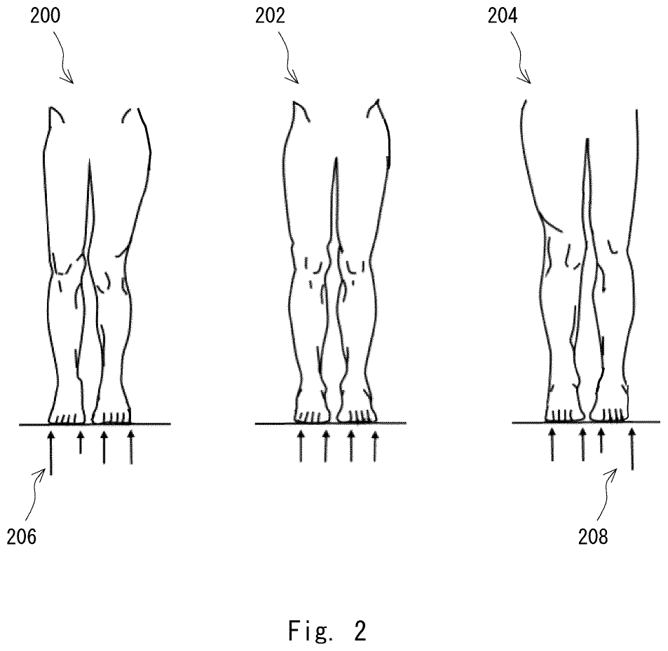

FIG. 1 shows an exemplary body image 100 which may be used to determine whether there is a rotation in the posture of the body (e.g., in the direction indicated by arrows 102 with reference to an upright reference axis line 104, such that the upper body is rotated in a clockwise direction indicated by the arrow 102 about the reference axis line 104). FIG. 2 shows images 200, 202 and 204 depicting a change in pressure through feet of a person having pelvic rotation, for example via use of a pressure mat to detect pressure points under the feet. Image 200 shows increased pressure at arrow 206 due to, for example, a clockwise rotation of the pelvis, image 200 shows a neutral pelvis position in which pressure through the feet is equal, and image 204 shows increased pressure at arrow 208 due to, for example, an anticlockwise rotation of the pelvis. These images of FIGS. 1 and 2 may be used to analyse shifting and tilting of a body part of a person. However, it is also beneficial to determine rotation of a body part, so that a cause of a shift or tilt can be identified. A body part can be better analysed to determine how good posture can be obtained via corrective training, such that an appropriate postural control training program can be recommended.

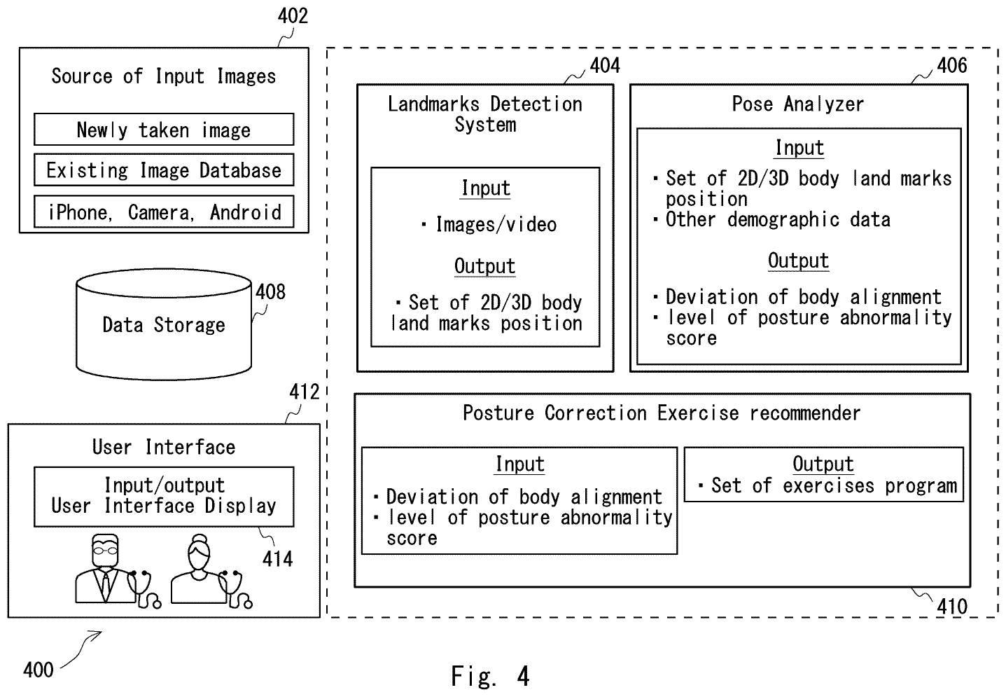

Further, there is still lack for detection of anatomical landmarks, providing deviation and postural assessment, and postural control training recommender system for the holistic postural assessment of a person's recovery (e.g., from the detected bad posture) and early intervention (e.g., to prevent further regression of bad posture). Thus, an overall system for assessing standing posture and for providing a recommendation for posture correction training may include an anatomical landmarks detection system, a posture analysis system for calculating of deviation of body part for frontal/anterior, posterior and lateral of a person, and a posture abnormality scoring system. An example of the overall system is described in FIG. 4. These systems are advantageously able to indicate a deviation of a body part alignment from a reference line. For example, a posture abnormalities scoring system can advantageously identify a severity of a posture condition, and a posture correction training recommendation system can advantageously enable realignment of an incorrect posture.

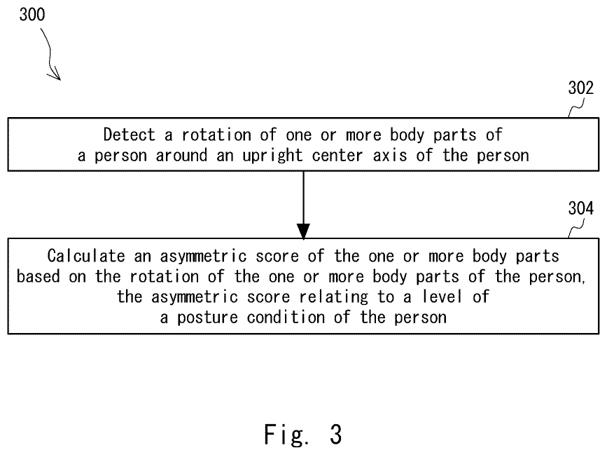

FIG. 3 shows a flow chart 300 illustrating a method for identifying a posture condition of a person according to various embodiments of the present disclosure. In step 302, a rotation of one or more body parts of a person around an upright center axis of the person is detected. In step 304, an asymmetric score of the one or more body parts is calculated based on the rotation of the one or more body parts of the person, the asymmetric score relating to a level of the posture condition of the person.

In an implementation, the method may further comprise detecting an angle of a first line connecting two body part positions of the person from an image of the person against a reference line, wherein the detection of the rotation of the one or more body parts of the person around the upright center axis of the person is based on the angle. The first line may connect a left side body part position and a right side body part position of a first body part of the person across a frontal plane and the reference line may comprise a second line connecting a left side body part position and a right side body part position of a second body part of the person across the frontal plane. The reference line may be a third line connecting another two body part positions of the person.

In an implementation, the method may comprise detecting a first distance of a body part position from a nearest point along the upright center axis of the person; wherein the detection of the rotation of the one or more body parts of the person and/or the calculation of the asymmetric score of the one or more body parts of the person is based on the first distance.

In an implementation, the method may comprise detecting a second distance of a body part position from one of (i) two body part positions of the person, (ii) a middle point between the two body part positions or (iii) a fourth line connecting the two body part positions, wherein the detection of the rotation of the one or more body parts of the person and/or the calculation of the asymmetric score of the one or more body parts of the person is based on the second distance.

In an implementation, the method may comprise detecting a plurality of body part positions of the person based on relative positions of a plurality of body parts in one or more images in which the person is detected, wherein each of plurality of body part positions corresponds to a body part of the person. Detecting the plurality of body part positions of the person may comprise estimating a body part position of the person based on one of the plurality of body part positions, wherein the plurality of body part positions of the person further comprises the estimated body part position.

In an implementation, the method may comprise receiving demographic data relating to the person; wherein the calculation of the asymmetric score is further based on the demographic data. The asymmetric score may be one of a plurality of asymmetric scores relating to a plurality of body parts, and the method may further comprise calculating the level of the posture condition of the person based on the plurality of asymmetric scores. The method may further comprise comparing each of the plurality of asymmetric scores with other asymmetric scores of the plurality of asymmetric scores; identifying one or more asymmetric scores having a higher score among the plurality of asymmetric scores; and identifying a set of posture correction programs based on a result of the identification.

FIG. 4 shows a block diagram of an apparatus 400 for identifying a posture condition of a person according to an embodiment of the present disclosure. In an implementation, the apparatus 400 may be generally described as a physical device comprising at least one processor and at least one memory including computer program code. The at least one memory and the computer program code are configured to, with the at least one processor, cause the physical device to perform the operations described in FIG. 3.

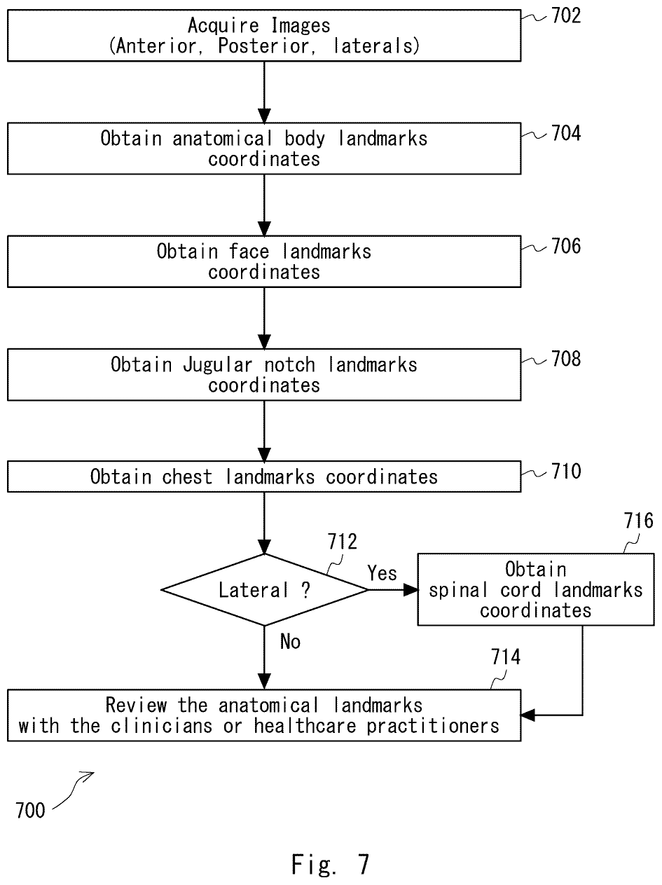

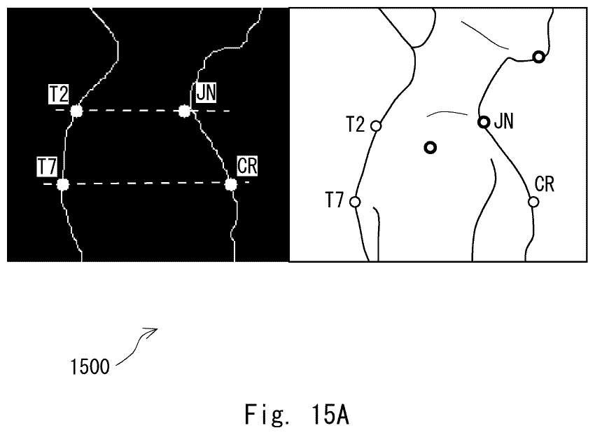

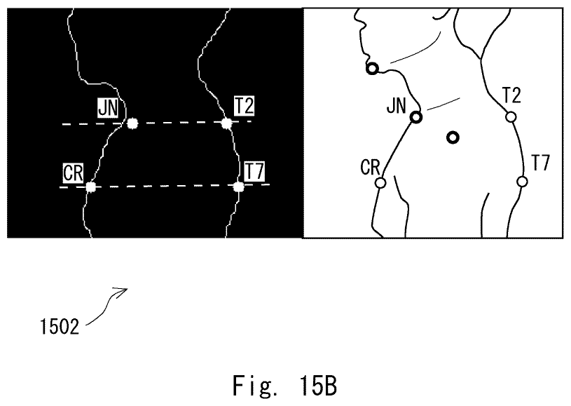

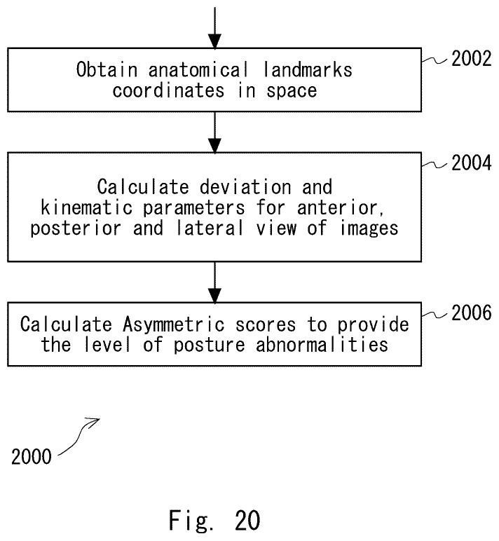

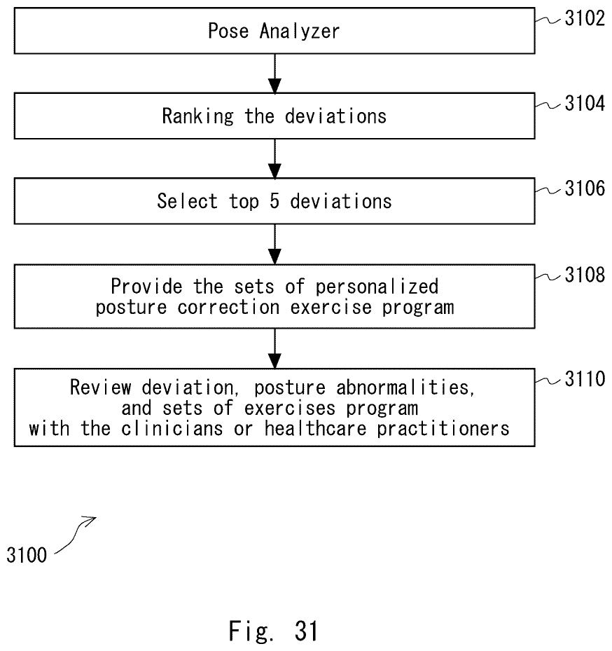

In an implementation, the apparatus 400 may receive input videos or images (e.g., video frames or images of an anterior view, a frontal view and/or a lateral view of a body of a person, or other similar image) from a source 402. For example, the input image or video may be a newly taken image or video, or one from an existing image or video database, or one taken by a camera or stored in a device such as a smartphone, camera, or other similar device. In an example, an input video may be deconstructed into a plurality of still video image frames so that each video image frame may be analysed by the apparatus 400. The apparatus 400 may comprise a landmarks detection system 404 configured to determine a set of two-dimensional and/or three-dimensional body landmarks position from the input videos or images, and a pose analyzer 406 configured to determine a deviation of body alignment and/or a level of posture abnormality score (e.g., asymmetric score) based on the set of two-dimensional and/or three-dimensional body landmarks position as well as other demographic data as input. In an example, the landmarks detection system 404 may be configured to determine whether an image is an anterior view, posterior view or lateral view of a body of a person for determining the landmark positions. The landmarks detection system 404 is further described in FIGS. 7-19, while the pose analyzer 406 is further described in FIGS. 20-30. The apparatus 400 may also comprise a posture correction exercise recommender 410 that is configured to recommend a suitable exercise or posture correction program based on the deviation of body alignment and/or a level of posture abnormality score (e.g., asymmetric score) as input. The posture correction exercise recommender 410 is further described in FIG. 31.

The apparatus 400 may be configured to display the received videos and/or images from the source 402 and/or inputs and outputs of the landmarks detection system 404, pose analyzer 406 and posture correction exercise recommender 410 in a user interface 412. A user of the apparatus 400 may also interact with the user interface 412 with data input via an input/output interface display 414. It should be appreciated that each of the landmarks detection system 404, pose analyzer 406 and posture correction exercise recommender 410 may be part of the apparatus 400, or a standalone device or part of another device and is in communication with the apparatus 400 through a connection. Such connection may be wired, wireless (e.g., via NFC communication, Bluetooth, etc.) or over a network (e.g., the Internet).

The apparatus may comprise a data storage 408 accessible by the apparatus 400 for storing videos and images from the source 402 as well as inputs and outputs of the landmarks detection system 404, pose analyzer 406 and posture correction exercise recommender 410. While it is shown in FIG. 4 that the data storage is part of the apparatus 400, it should be appreciated that the data storage 408 may not form part of the apparatus 400 and is in communication with the apparatus 400 through a connection. Such connection may be wired, wireless (e.g., via NFC communication, Bluetooth, etc.) or over a network (e.g., the Internet) or over a cloud server. In another implementation, each of the landmarks detection system 404, pose analyzer 406, posture correction exercise recommender 410 and the source 402 may comprise its own data storage for storing its input/output data.

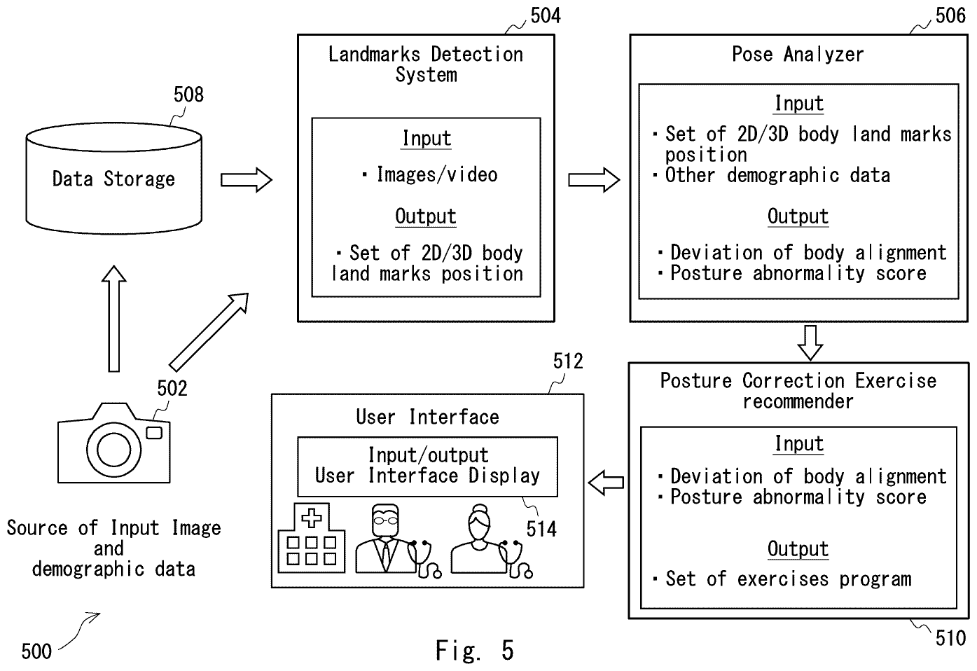

FIG. 5 shows an overall system workflow diagram 500 for identifying a posture condition of a person according to an embodiment of the present disclosure. In an implementation, a video or image (e.g., video frame or image of an anterior view, a frontal view and/or a lateral view of a body of a person, or other similar image) may be received from a source (e.g., an image and/or video capturing apparatus, a database, the internet, or other similar sources) and stored in data storage 508 before being used by landmarks detection system 504 as input, or directly sent to the landmarks detection system 504 as input without going through the data storage 508. The landmarks detection system 504 may determine a set of two-dimensional and/or three-dimensional body landmarks position from the input video or image, and a pose analyzer 506 may determine a deviation of body alignment and/or a level of posture abnormality score (e.g., asymmetric score) based on the set of two-dimensional and/or three-dimensional body landmarks position as well as other demographic data as input. A posture correction exercise recommender 410 may recommend a suitable exercise or posture correction program based on the deviation of body alignment and/or a level of posture abnormality score (e.g., asymmetric score) as input. The recommended exercise or posture correction program may then be displayed in an input/output user interface display of a user interface 512.

In an implementation, a system for identifying a posture condition of a person may comprise the apparatus 400 and one or more image capturing apparatuses configured to capture one or more images of the person, wherein the one or more images comprises an image of the person across a frontal plane and/or an image of the person across a sagittal plane.

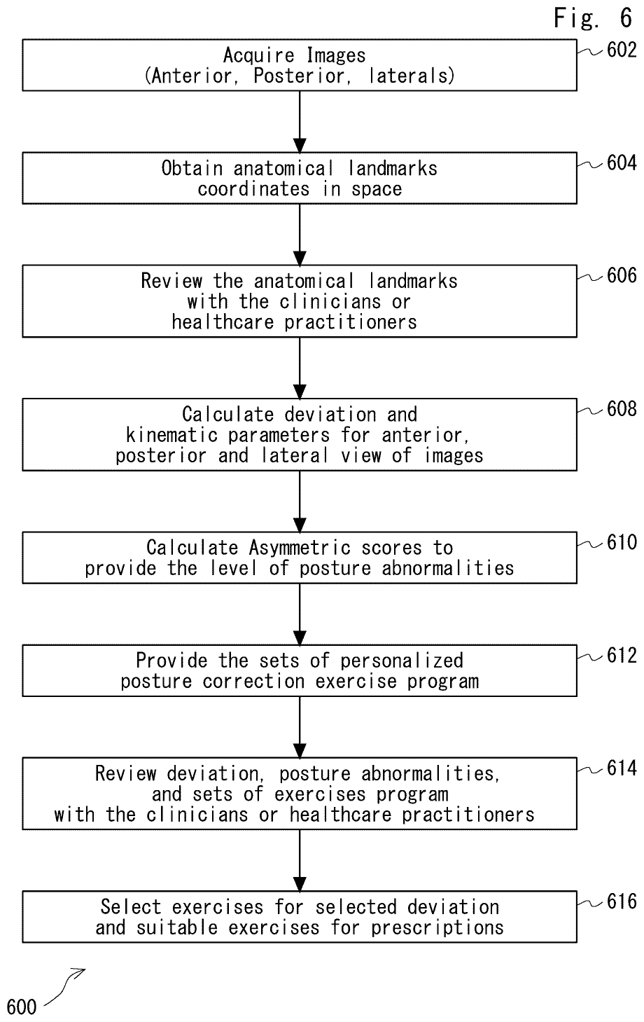

FIG. 6 shows a flowchart 600 for identifying a posture condition of a person according to an embodiment of the present disclosure. In a step 602, images of anterior, posterior and laterals of a body of a person may be acquired. In an example, it may be determined whether an acquired image is an anterior, posterior or lateral view of a body of a person. In an example where an input video is acquired, the video may be deconstructed into a plurality of still video image frames, and it may be determined whether each video image frame is an anterior, posterior or lateral view of a body of a person. In a step 604, anatomical landmarks (e.g., space coordinates for each landmark) for each body part in the images may be obtained. In a step 606, the obtained anatomical landmarks may be reviewed with clinicians or healthcare practitioners. In a step 608, deviation and kinematic parameters for anterior, posterior and lateral view of images are calculated. In a step 610, asymmetric scores to provide a level of posture abnormalities are calculated. In a step 612, one or more sets of personalized posture correction exercise programs are provided. In a step 614, the deviation, posture abnormalities, and the one or more sets of exercises program are reviewed with the clinicians or healthcare practitioners. In a step 616, one or more exercises for a selected deviation (e.g., based on the asymmetric scores) and suitable exercises are selected as a prescription for the person.

FIG. 7 shows a flowchart 700 for a landmarks detection system according to an embodiment of the present disclosure. In a step 702, images of anterior, posterior and laterals of a body of a person may be acquired. In an example, it may be determined whether an acquired image is an anterior, posterior or lateral view of a body of a person. In an example where an input video is acquired, the video may be deconstructed into a plurality of still video image frames, and it may be determined whether each video image frame is an anterior, posterior or lateral view of a body of a person. In a step 704, anatomical landmarks (e.g., space coordinates for each landmark) for each body part in the images may be obtained. In a step 706, face landmark coordinates are obtained. In a step 708, landmark coordinates for the jugular notch of the person are obtained. In a step 710, landmark coordinates for the chest are obtained. In a step 712, it is determined whether the acquired image is a lateral view of the body of the person. If it is not determined to be case, the process proceeds to step 714 where the obtained anatomical landmarks may be reviewed with clinicians or healthcare practitioners, and then the process ends. Otherwise, the process proceeds to step 716 in which spinal cord landmark coordinates are obtained, and then to step 714 where the obtained anatomical landmarks (e.g., including the spinal cord landmark coordinates) may be reviewed with clinicians or healthcare practitioners, and then the process ends.

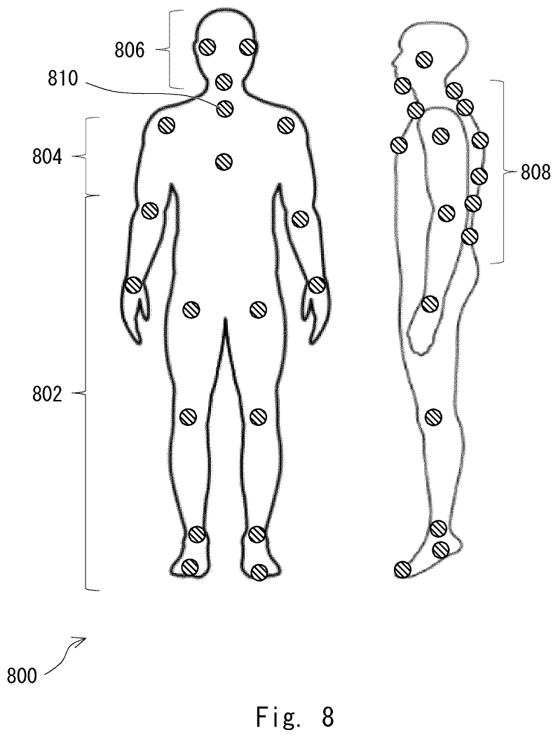

FIG. 8 shows an exemplary illustration 800 for landmarks detection according to an embodiment of the present disclosure. For example, with reference to flowchart 700, landmark coordinates for each body part in section 802 may be obtained in step 704. Landmark coordinates for face section 806 may be obtained in step 706. Landmark coordinates for chest section 804 may be obtained in step 710. Spinal landmark coordinates in spinal section 808 (e.g., which can be seen from a lateral view of a body of a person) may be obtained in step 716. Further, a landmark coordinate 810 denoting the jugular notch may be obtained in step 708.

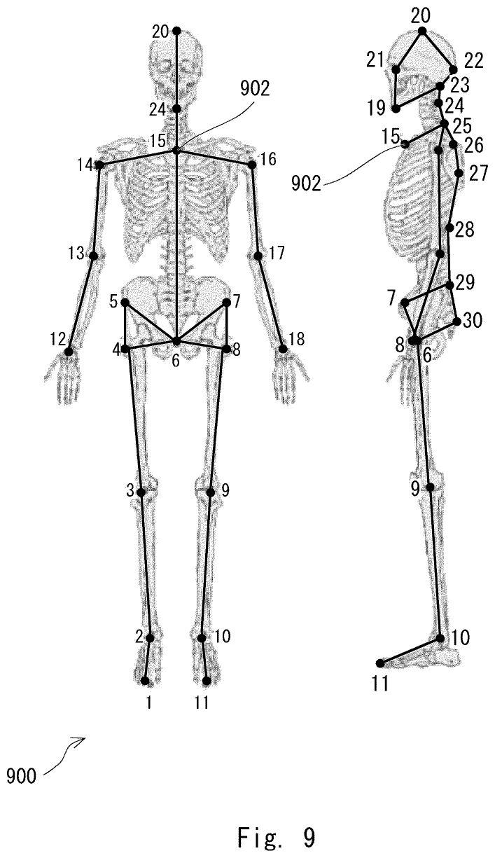

In an example, open source body and face landmark detection engines such as MediaPipe holistic engine may be utilized to obtain the required anatomical landmark coordinates. However, open source engines typically cannot provide some of the landmarks required by clinicians as shown in landmarks FIG. 900 of FIG. 9. For example, the landmark for the jugular notch as shown by reference 902 is typically manually provided by the clinicians. It will be appreciated that the required anatomical landmark coordinates may also be obtained by other non-open source proprietary landmark detection engines.

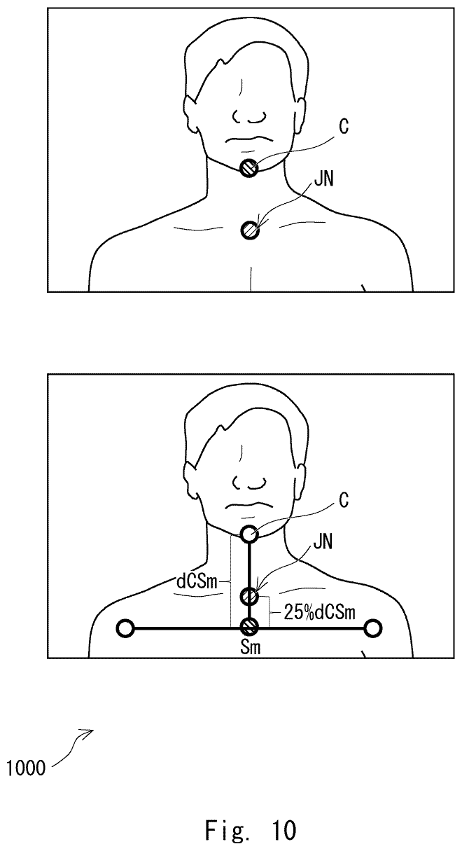

FIG. 10 shows an illustration 1000 for detection of jugular notch (JN) from a front view of a body of a person (e.g., an image of the person across a frontal plane) according to an embodiment of the present disclosure. A position of each of a chin, left shoulder and right shoulder of the person may be determined based on a plurality of body part positions and/or face part positions that are detected, for example, in steps 704 and 706 of flowchart 700. For example, landmark coordinate of the chin (C) may be defined as C: [Cx, Cy] (obtained in, for example, step 706 of flowchart 700), and landmark coordinate of the shoulder midpoint (Sm) may be defined as Sm:[Smx, Smy](e.g., identifying a midpoint position between the positions of the left and right shoulders, based on landmark coordinates corresponding to the left and right shoulders of the person obtained in, for example, step 704 of flowchart 700). Further, a distance from the position of the chin to the shoulder midpoint position may be calculated and defined as dCSm. The position of the jugular notch of the person may then be estimated based on the position of the chin and the calculated distance. For example, the landmark coordinate of the jugular notch may be defined as JN: [JNx, JNy]. Thus, based on the landmark coordinates of the chin, shoulders and midpoint of the shoulders, it can be determined that JNx is equivalent to Smx, and JNy is equivalent to Smy−25% dCSm.

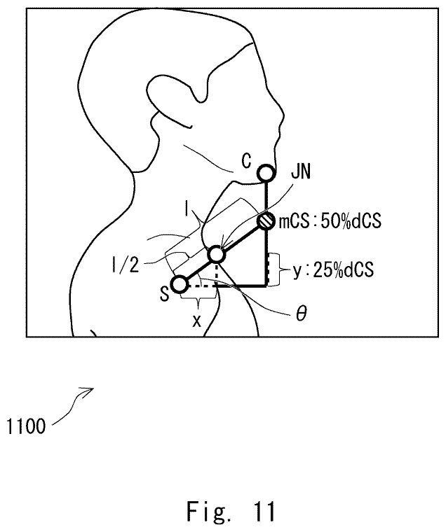

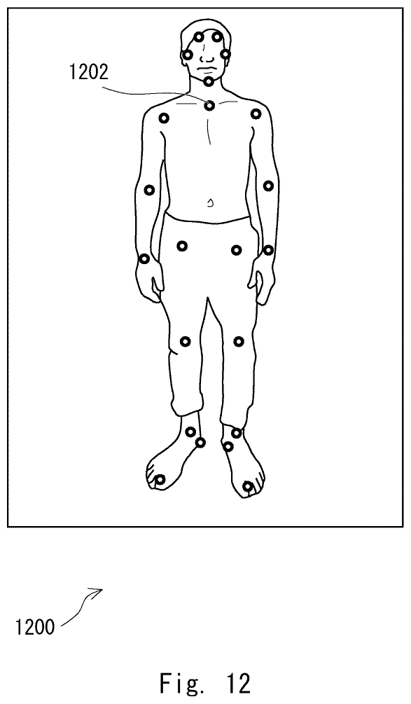

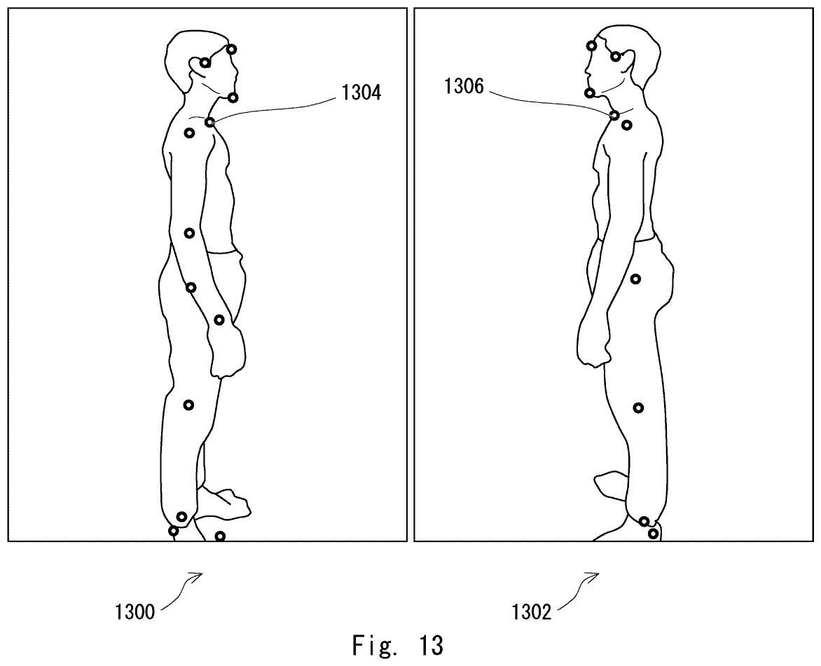

Further, it is also possible to determine a position of the jugular notch from a side view of the body of the person (e.g., an image of the person across a sagittal plane) as shown in illustration 1100 of FIG. 11. A position of each of a chin and left or right shoulder (depending on the side of the person that the image shows) of the person may be determined based on a plurality of body part positions that are detected, for example, in steps 704 and 706 of flowchart 700. A landmark coordinate of the shoulder (S) may be defined as S:[Sx, Sy](e.g., based on landmark coordinates corresponding to the left or right shoulder of the person obtained in, for example, step 704 of flowchart 700). A midpoint position along a vertical line that starts from the position of the chin (e.g., landmark coordinates corresponding to the position of the chin obtained in, for example, step 706 of flowchart 700) and ends at a horizontal line passing through the position of the shoulder may be identified. The vertical distance from the position of the chin (C) to the horizontal line may be defined as dCS, 50% of this vertical distance (e.g., a distance from the position of the chin to the midpoint position) may be calculated and defined as mCS, a distance from the shoulder and the midpoint may be calculated and defined as 1, and 0 may be calculated and defined as an angle between a line from the position of the shoulder to the midpoint position and the horizontal line passing through the position of the shoulder (S). Based on the calculated distance I and calculated angle θ, the jugular notch (JN) can be detected and defined as JN: [JNx, JNy], in which JNx is equivalent to Sx±x, where x=cos(θ)*½, and JNy is equivalent to Sy−y, where y=25% dCS. Based on these techniques and calculations, it is possible to identify the jugular notch based on front view images (e.g., such as jugular notch landmark 1202 from exemplary front view image 1200 of FIG. 12 respectively) as well as from lateral view images (e.g., such as jugular notch landmarks 1304 and 1306 from exemplary lateral view images 1300 and 1302 of FIG. 13 respectively)

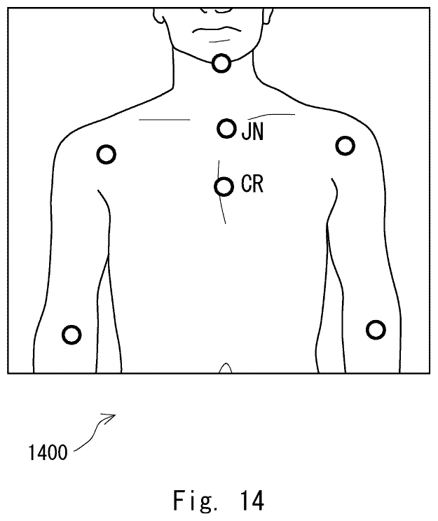

FIG. 14 shows a frontal view for identification of a central ray (CR) position based on a detected jugular notch (JN) according to an embodiment of the present disclosure. Based on the jugular notch (JN), a central ray (CR) position may be computed, wherein the central ray position may be 3-4 inches (e.g., 8-10 cm) below the position of the jugular notch. A mirror image of the CR position or mid chest or T7 may be detected using right lateral view 1500 and/or left lateral view 1502 of a body of a person for detection of a central ray (CR) position, chest and mid-chest (e.g., T7) based on the jugular notch (JN). For example, a landmark used for positioning the central ray (CR) is at T7 (e.g., the mid thorax). The level of the T7 may be 3-4 inches (e.g., 8-10 cm) below the jugular notch.

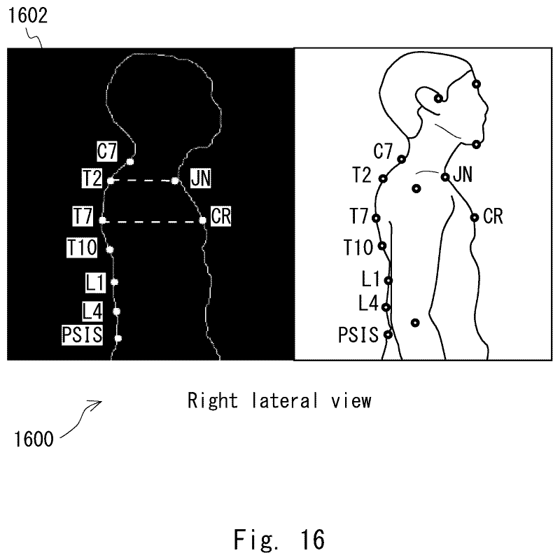

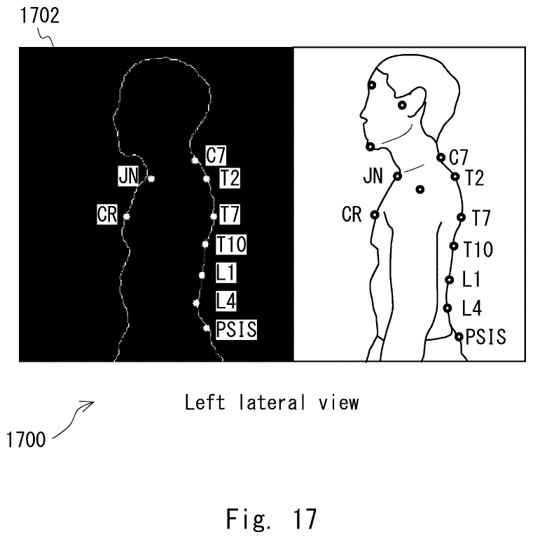

FIGS. 16 and 17 show respectively a right lateral view 1600 and left lateral view 1700 for estimation of cervical (C7), thoracic (T2, T7, T10), lumbar (L1 and L4) and sacral (S2) vertebrae positions of a spinal cord according to an embodiment of the present disclosure. A position of a vertebrae along a spinal cord of the person may be estimated based on the estimated position of the mid chest of the person and a relative distance between each vertebrae along the spinal cord. For example, based on the approximate distance between T7 and C7, and prior research on the ratio of thoracic and posterior superior iliac spine (PSIS) by Ernst et al., C7, T2, T7, T10, L1, L4 and S2 (PSIS) positions may be estimated using the lateral view images 1600 and 1700. In an example, a flexible ruler may also be placed on a back of a person for determining the distances between the various segments of the spinal cord. The estimated distance may be utilized for calculating a percentage position on a distance throughout the segments from C7 to PSIS.

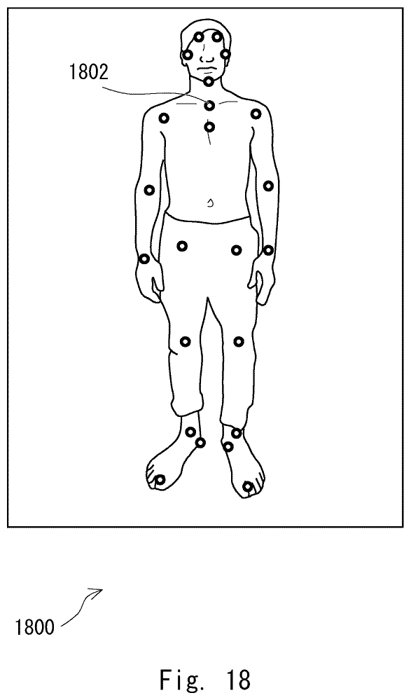

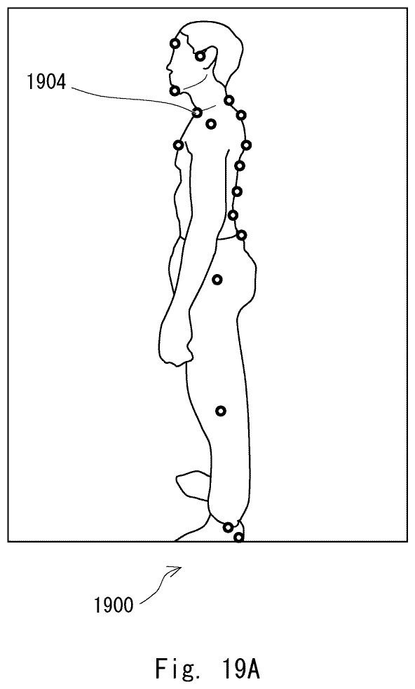

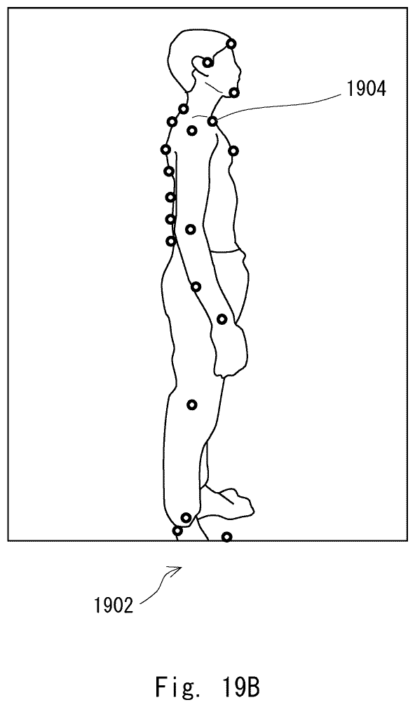

FIGS. 18, 19A and 19B show respectively results of the landmarks detection system from an anterior/frontal view image 1800, left lateral view image 1900 and right lateral view image 1902 according to an embodiment of the present disclosure, each landmark denoting a body part and represented by a dot on the body of the person in the images. For example, the jugular notch of the person is denoted by a landmark 1802 in the image 1800, and by a landmark 1904 in images 1900 and 1902. Further results of the landmarks detection system are also shown in body edge image 1602 of FIG. 16 and body edge image 1702 of FIG. 17 in which, based on detected landmarks for a whole body of a person in for example images 1900 and 1902, the landmarks that are detected for the spinal cord may be selected and displayed on a body edge image 1702 and 1602 respectively e.g., the detected spinal cord landmarks being shown as white dots in the body edge images 1602 and 1702.

FIG. 20 shows a flow chart 2000 illustrating for a posture analyzer according to an embodiment of the present disclosure. In a step 2002, anatomical landmark coordinates are obtained, e.g., from one or more images depicting anterior, posterior and lateral views of a body of a person. For example, the landmark coordinates may be obtained as input from the landmark detection system 404. Further, other demographic data relating to the person may also be obtained. In a step 2004, deviation and kinematic parameters are obtained for the anterior, posterior and lateral views. In an example, the deviation and kinematic parameters may be based on the landmark coordinates, and may also be based on other demographic data relating to the person. In a step 2006, asymmetric scores to provide a level representative of one or more posture abnormalities are calculated.

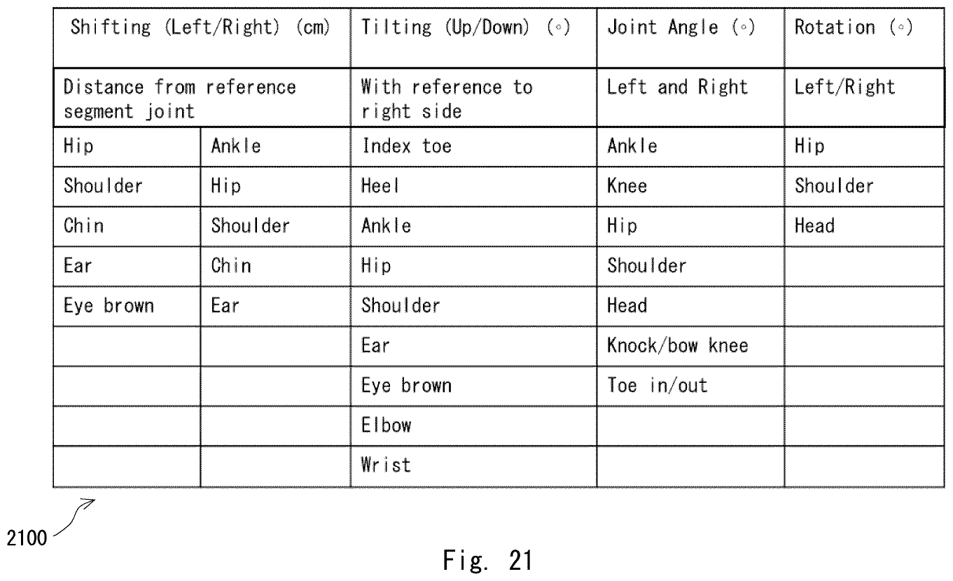

FIG. 21 shows a table for calculation of deviation and kinematic parameters for anterior view of body images (e.g., as calculated in step 2004 of flowchart 2000) according to an embodiment of the present disclosure. Based on anterior view of a body of a person, deviation and kinematic parameters may be calculated for various posture abnormalities such as left or right shifting (e.g., in cm), up or down tilting (e.g., in degrees), joint angles (e.g., an angle in degrees between a first line connecting two body parts and a second line connecting another two body parts), and rotation (e.g., in degrees). In an implementation, calculations for shifting of a body part may be based on a distance of the midpoint of the body part from a midpoint of a reference segment joint such as between the hip and ankle, between the shoulder and hip, between the chin and shoulder, between the ear and chin, between the eye brow and the ear, and other similar distances. In another implementation, shifting of a body part may be calculated by computing a distance of a midpoint of the body part from a reference plumb line or a center of gravity line. In an implementation, calculations for tilting of a body part may be with reference to a body part on the right or left side of a body of a person, such as the index toe, heel, ankle, hip, shoulder, ear, eye brow, elbow, wrist, and other similar body part.

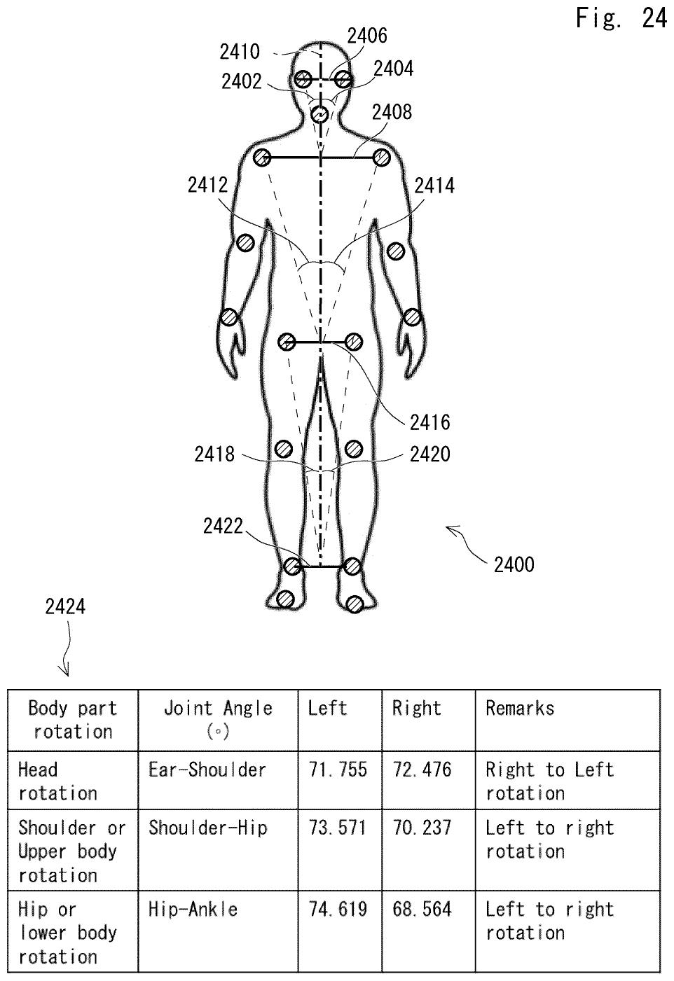

In another implementation, calculations for joint angle may be with reference to a first line connecting a left and right body part, and a second line connecting another left and right body part (e.g., a line connecting left and right shoulder, left and right sides of the hip, left and right knees, left and right toes, or other similar body parts). For example, a left joint angle and a right joint angle may be obtained by calculating an angle on a left side and a right side respectively of a straight third line cutting through a mid-point of the first line and a mid-point of the second line, with reference to the left and right body part connected by the first or second line. More details for the joint angle will be shown in FIG. 24. Further, calculations for rotation of a body part (e.g., hip, shoulder, ear, or other similar body part) may be based on the calculated left and right joint angles.

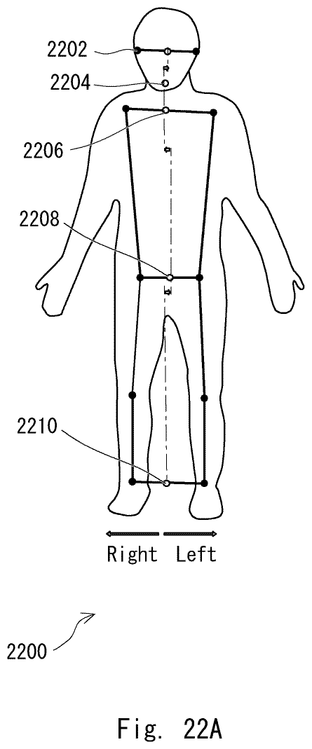

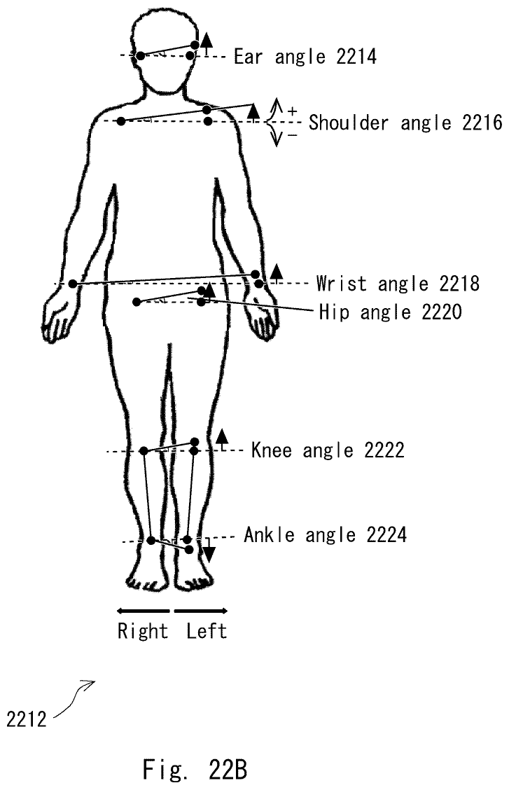

Based on a frontal view of a body of a person in a standing posture, the following analysis may be provided. For example, left and right shifting may be determined based on a deviation of distance of a body segment from a reference body segment, such as a distance of the hip from the ankle, distance of the shoulder from the hip, distance from the ear to the chin, eye brow distance from the ear, chin distance from the shoulder, and other similar distances. In the present disclosure, “−” means shifted to the left side of the body, and “+” mean shifted to the right side of the body. For example, referring to frontal view image 2200 of FIG. 22A, it may be determined that ear position (denoted by reference 2202) is shifted 0.2 cm to the left with reference to the jaw, jaw position (denoted by reference 2204) is shifted 0.05 cm to the right with reference to the shoulder, shoulder position (denoted by reference 2206) is shifted to 0.2 cm to the left with reference to the hip, hip position (denoted by reference 2208) is shifted 0.2 cm to the left from the ankle, and ankle position is denoted by reference 2210.

In another example, angle of left and right tilting may be determined based on how a position of a body joint (e.g., ankle, knee, hip, shoulder, ear angle) is tilted from a reference side. In the present disclosure, “−” means tilted down from a reference plane, and “+” mean tilted up from the reference plane. For example, referring to frontal view image 2212 of FIG. 22B, tilting of the neck may be determined based on ear angle 2214, tilting of the shoulder may be determined based on shoulder angle 2216, tilting of the wrist may be determined based on wrist angle 2218, tilting of the hip may be determined based on hip angle 2220, tilting of the knee may be determined based on knee angle 2222, tilting of the ankle may be determined based on ankle angle 2224.

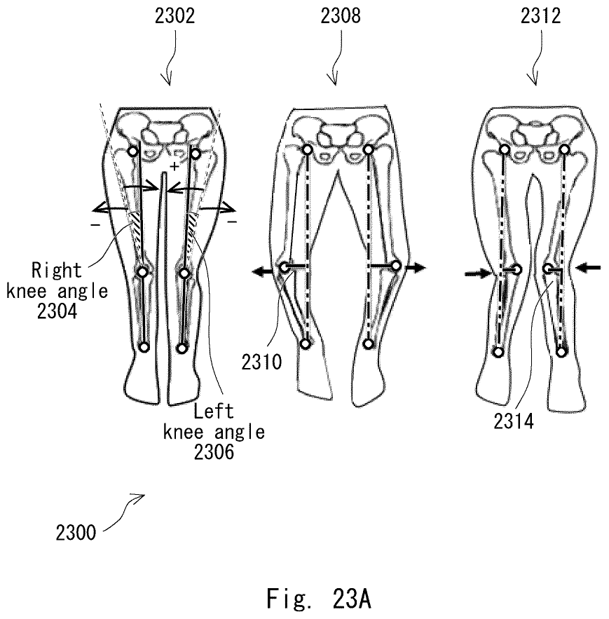

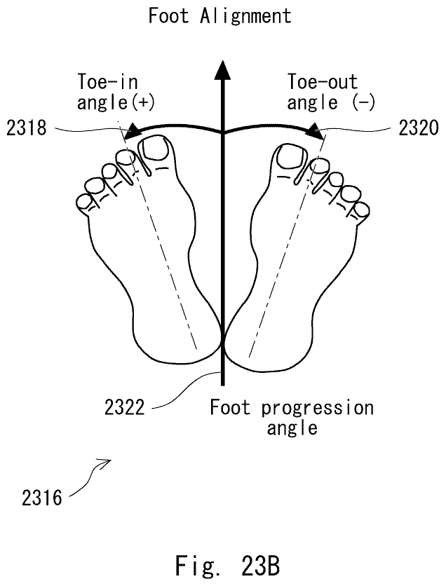

Lower extremities analysis may also be performed from a frontal view to determine a knock or bow knee, referring to illustration 2300 of FIG. 23A, (e.g., based on right knee angle 2304 and left knee angle 2306 of image 2302), “−” means knock knee and “+” means bow knee. Further referring to illustrations of 2308 and 2312 of FIG. 23A, it may be determined whether a knock knee or bow knee exists (and also a degree of severity of the knock knee or bow knee), for example based on a distance 2310 between the right knee and a line connecting the right hip and the right ankle for a bow knee image 2308, or based on a distance 2314 between the left knee and a line connecting the left hip and the left ankle for a knock knee image 2312. Further, referring to illustration of FIG. 23B, toe in and toe out position of the foot can be determined. For example, referring to illustration 2316 of FIG. 23B, foot alignment of a person may be determined based on a toe angle with reference to a reference line 2322, Toe angle positive (+) means toe in angle 2318 or toe angle negative (−) means toe-out angle 2320.

FIG. 24 shows an illustration 2400 for determining body part rotation according to an embodiment of the present disclosure. For example, head rotation may be based on analysis of ear-shoulder joint angles 2402 and 2404 calculated from a first line 2406 connecting the ears and a second line 2408 connecting the shoulders, with reference from a third line 2410. Shoulder or upper body rotation may be based on analysis of shoulder-hip joint angles 2412 and 2414 calculated from a first line 2408 connecting the shoulders and a second line 2416 connecting the hips, with reference from a third line 2410. Further, hip or lower body rotation may be based on analysis of hip-ankle joint angles 2418 and 2420 calculated from a first line 2416 connecting the hips and a second line 2422 connecting the ankles, with reference from a third line 2410. The obtained joint angles may be analysed as shown in table 2424. For example, if the left ear-shoulder joint angle is smaller than the right ear-shoulder joint angle, it indicates that a right-to-left head rotation exists. If the left shoulder-hip joint angle is larger than the right shoulder-hip joint angle, it indicates that a left-to-right shoulder or upper body rotation exists. Further, if the left hip-ankle joint angle is larger than the right hip-ankle joint angle, it indicates that a left-to-right hip or lower body rotation exists.

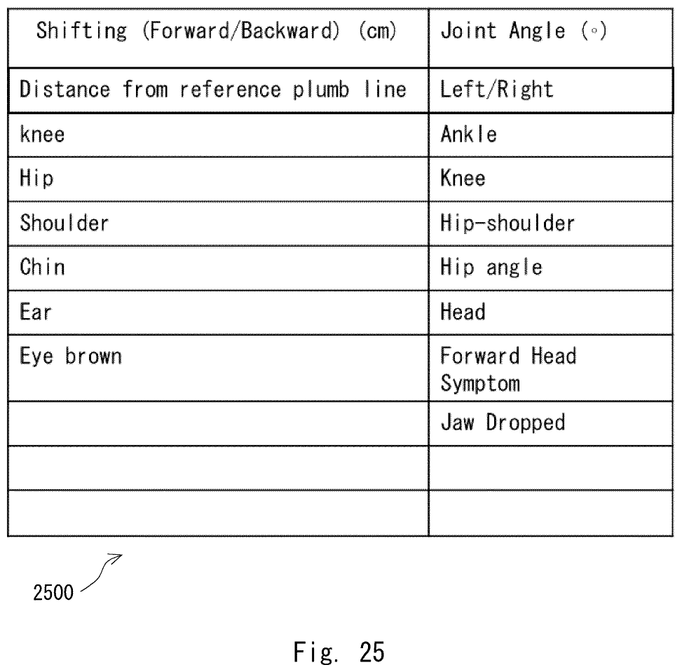

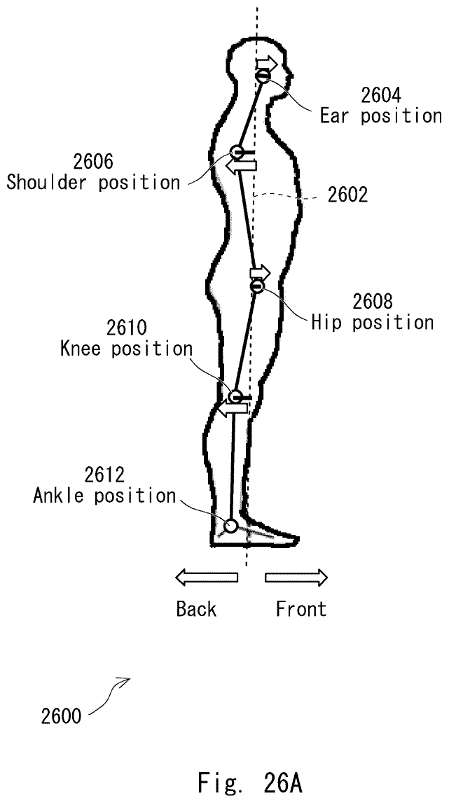

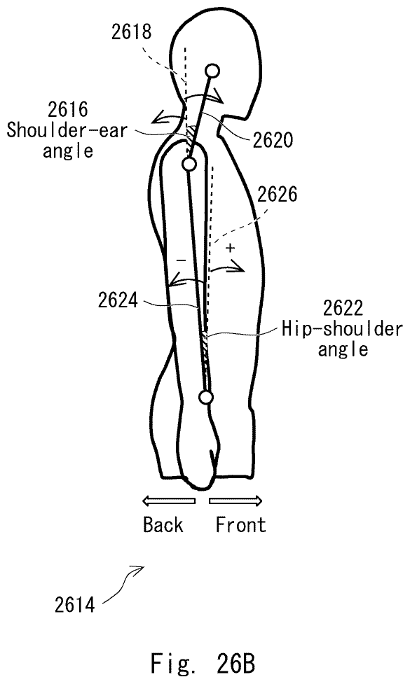

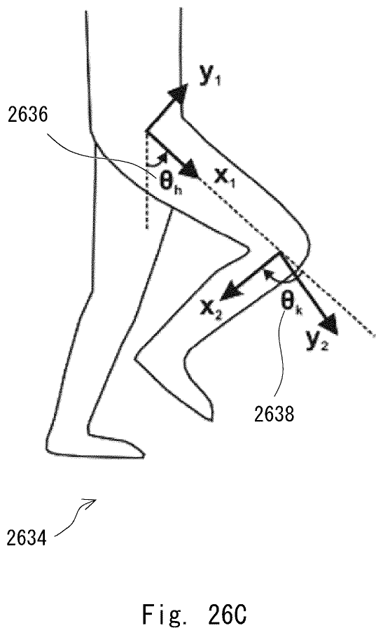

FIG. 25 shows a table 2500 for calculation of deviation and kinematic parameters for lateral view of body images according to an embodiment of the present disclosure. For example, forward and backward shifting of body parts such as the knee, hip, shoulder, chin, ear and eye brow may be determined based on a distance from a reference plumb line. Referring to lateral view image 2600 of FIG. 26A, with reference to plumb line 2602 which runs vertically through 2 cm anterior from the ankle position 2612, it is possible to determine that ear position (denoted by reference 2604) is shifted 2.0 cm forward, shoulder position (denoted by reference 2606) is shifted 3.0 cm backward, hip position (denoted by reference 2608) is shifted 0.50 cm forward, and knee position (denoted by reference 2610) is shifted 3.0 cm backward.

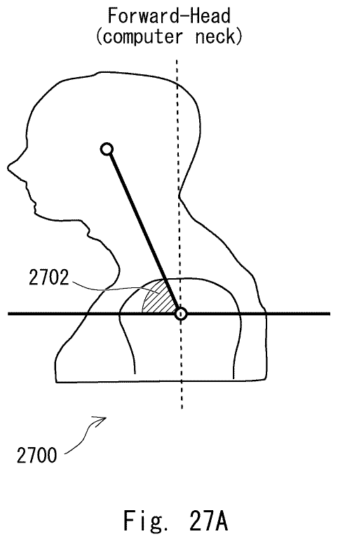

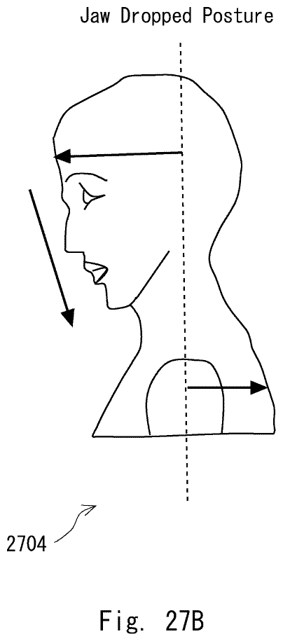

Further, joint angles for the ankle, knee, hip-shoulder, hip angle, head, as well as for forward head symptom and jaw dropped symptom may be measured in degrees with reference to a reference line. For example, referring to lateral view image 2614 of FIG. 26B, a head or shoulder-ear angle 2616 may be determined with reference to line 2620 connecting the ear to the shoulder as well as vertical line 2618. Hip-shoulder angle 2622 may be determined with reference to line 2624 connecting the hip and shoulder as well as vertical line 2626. In another example shown in lateral view image 2634 of FIG. 26C, parameters such as hip angle 2636 and knee angle 2638 may be determined. Furthermore, joint angle 2702 for forward head symptom may be determined based on lateral view image 2700 of FIG. 27A, and corresponding deviation and kinematic parameters may also be calculated from jaw-dropped posture lateral image 2704 of FIG. 27B.

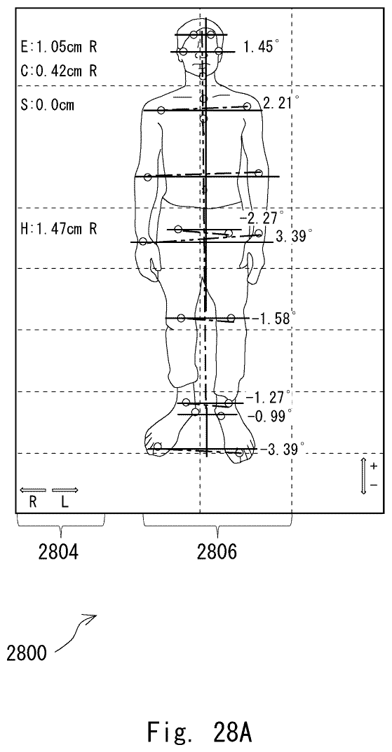

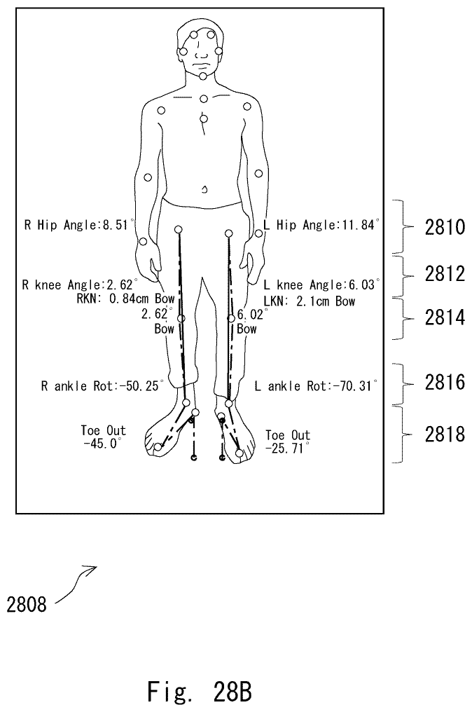

FIGS. 28A and 28B show exemplary illustrations 2800 and 2808 of posture analyzer output based on a front body view according to an embodiment of the present disclosure. For example in the front body view of illustration 2800, annotations indicating left and right shifting and tilting of each body joint (e.g., annotations 2804 indicating shifting and annotations 2806 indicating joint angles of respective body parts) are displayed over the body image. Further, in the front body view of illustration 2808, annotations indicating left and right lower extremities analysis (e.g., annotations 2810 indicating hip angles, annotations 2812 indicating knee angles, annotations 2814 indicating bow knee, annotations 2816 indicating ankle references and annotations 2818 indicating toe-out symptoms) are displayed over the body image.

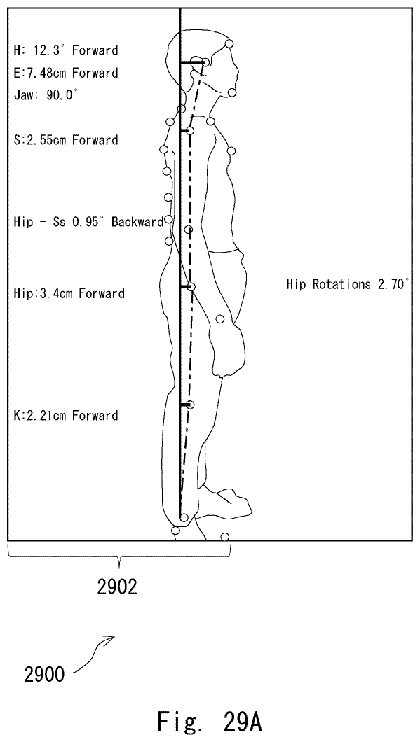

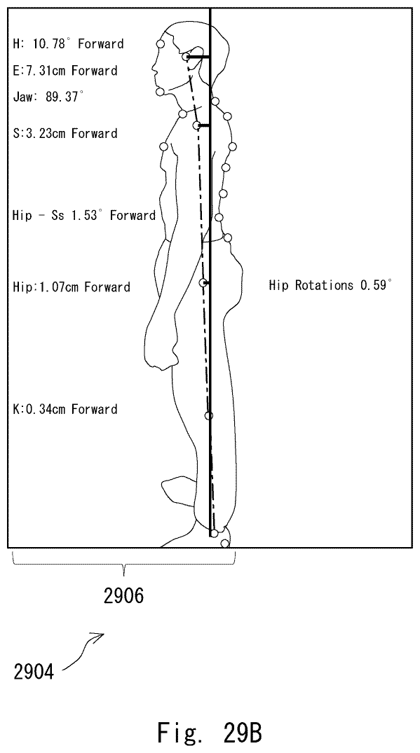

FIGS. 29A and 29B show exemplary illustrations 2900 and 2904 of posture analyzer output based on a right side and left side body view respectively according to an embodiment of the present disclosure. For example in the right side body view of illustration 2900, annotations 2902 indicating right shifting from plumb line specific joint angle are displayed over the body image. Further, in the left side body view of illustration 2904, annotations 2906 indicating left shifting from plumb line specific joint angles are displayed over the body image.

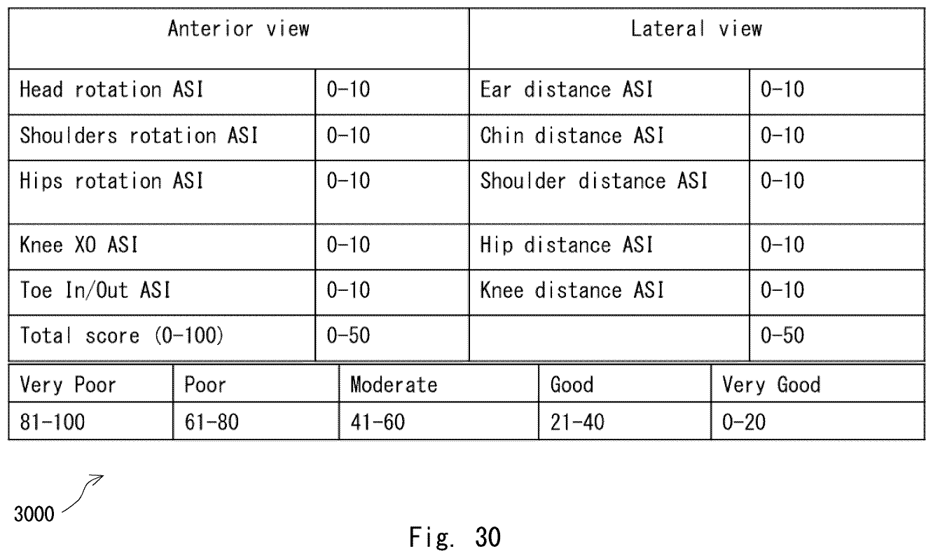

FIG. 30 shows a table 3000 for calculation of asymmetric scores to provide a level of posture abnormalities according to an embodiment of the present disclosure. For example, an asymmetric index (ASI) may be calculated for each of a head rotation, shoulders rotation, hips rotation, knock or bow knee, toe-in or toe-out symptom, ear distance, chin distance, shoulder distance, hip distance and knee distance. Each ASI score may be calculated via formula Asymmetry Index (ASI)=10*(left−right)/(left+right), and may range from a value of 0-10. The ASI scores of the above mentioned body alignments are summed, allowing a range of overall score between 0 and 100. A higher ASI score indicates a more severely displaced posture, and more emphasis may thus be placed on the more severe postures for correction and rehabilitation.

FIG. 31 shows a flow chart 3100 illustrating for a posture correction exercise recommender according to an embodiment of the present disclosure. In a step 3102, pose analyzer (e.g., pose analyzer 406/506) may be utilized to obtain deviation of all concerned body alignments as well as calculate a level of posture abnormality score (e.g., ASI score). In a step 3104, the obtained deviations are ranked based on the ASI score. In a step 3106, the top 5 deviations (e.g., top 5 deviations with the top 5 highest ASI score) are selected. In a step 3108, one or more sets of a personalized posture correction exercise program are provided based on the top 5 deviations (e.g., recommendation of personalized posture correction exercise program for correcting the top 5 deviations). For example, exercises such as 3-position toe raises, 45 degree neck stretch, hip rotations, press ups, shoulder blade press, tricep stretch and other similar exercises may be recommended based on the ASI score for the personalized posture correction exercise program. In a step 3110, the deviation, posture abnormalities, and sets of exercises program are reviewed with the clinicians or healthcare practitioners.

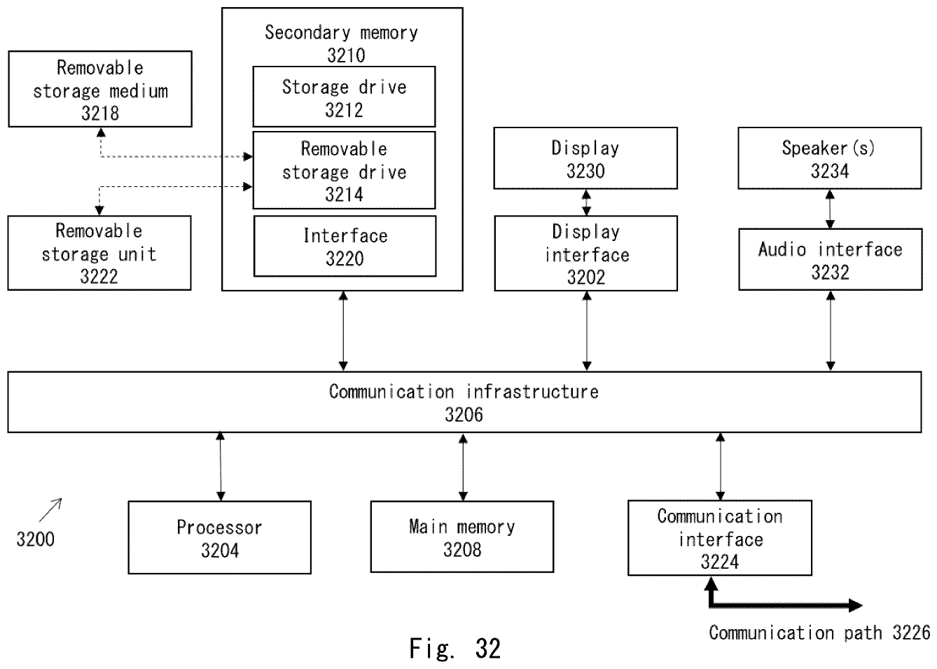

FIG. 32 depicts an exemplary computing device 3200, hereinafter interchangeably referred to as a computer system 3200, where one or more such computing devices 3200 may be used to execute the method of FIG. 3. The exemplary computing device 3200 can be used to implement the apparatus 400 shown in FIG. 4. The following description of the computing device 3200 is provided by way of example only and is not intended to be limiting.

As shown in FIG. 32, the example computing device 3200 includes a processor 3204 for executing software routines. Although a single processor is shown for the sake of clarity, the computing device 3200 may also include a multi-processor system. The processor 3204 is connected to a communication infrastructure 3206 for communication with other components of the computing device 3200. The communication infrastructure 3206 may include, for example, a communications bus, cross-bar, or network.

The computing device 3200 further includes a main memory 3208, such as a random access memory (RAM), and a secondary memory 3210. The secondary memory 3210 may include, for example, a storage drive 3212, which may be a hard disk drive, a solid state drive or a hybrid drive and/or a removable storage drive 3214, which may include a magnetic tape drive, an optical disk drive, a solid state storage drive (such as a USB flash drive, a flash memory device, a solid state drive or a memory card), or the like. The removable storage drive 3214 reads from and/or writes to a removable storage medium 3218 in a well-known manner. The removable storage medium 3218 may include magnetic tape, optical disk, non-volatile memory storage medium, or the like, which is read by and written to by removable storage drive 3214. As will be appreciated by persons skilled in the relevant art(s), the removable storage medium 3218 includes a computer readable storage medium having stored therein computer executable program code instructions and/or data.

In an alternative implementation, the secondary memory 3210 may additionally or alternatively include other similar means for allowing computer programs or other instructions to be loaded into the computing device 3200. Such means can include, for example, a removable storage unit 3222 and an interface 3220. Examples of a removable storage unit 3222 and interface 3220 include a program cartridge and cartridge interface (such as that found in video game console devices), a removable memory chip (such as an EPROM or PROM) and associated socket, a removable solid state storage drive (such as a USB flash drive, a flash memory device, a solid state drive or a memory card), and other removable storage units 3222 and interfaces 3220 which allow software and data to be transferred from the removable storage unit 3222 to the computer system 3200.

The computing device 3200 also includes at least one communication interface 3224. The communication interface 3224 allows software and data to be transferred between computing device 3200 and external devices via a communication path 3226. In various embodiments of the inventions, the communication interface 3224 permits data to be transferred between the computing device 3200 and a data communication network, such as a public data or private data communication network. The communication interface 3224 may be used to exchange data between different computing devices 3200 which such computing devices 3200 form part an interconnected computer network. Examples of a communication interface 3224 can include a modem, a network interface (such as an Ethernet card), a communication port (such as a serial, parallel, printer, GPIB, IEEE 1394, RJ45, USB), an antenna with associated circuitry and the like. The communication interface 3224 may be wired or may be wireless. Software and data transferred via the communication interface 3224 are in the form of signals which can be electronic, electromagnetic, optical or other signals capable of being received by communication interface 3224. These signals are provided to the communication interface via the communication path 3226.

As shown in FIG. 32, the computing device 3200 further includes a display interface 3202 which performs operations for rendering images to an associated display 3230 and an audio interface 3232 for performing operations for playing audio content via associated speaker(s) 3234.

As used herein, the term “computer program product” may refer, in part, to removable storage medium 3218, removable storage unit 3222, a hard disk installed in storage drive 3212, or a carrier wave carrying software over communication path 3226 (wireless link or cable) to communication interface 3224. Computer readable storage media refers to any non-transitory, non-volatile tangible storage medium that provides recorded instructions and/or data to the computing device 3200 for execution and/or processing. Examples of such storage media include magnetic tape, CD-ROM, DVD, Blu-ray Disc, a hard disk drive, a ROM or integrated circuit, a solid state storage drive (such as a USB flash drive, a flash memory device, a solid state drive or a memory card), a hybrid drive, a magneto-optical disk, or a computer readable card such as a PCMCIA card and the like, whether or not such devices are internal or external of the computing device 3200. Examples of transitory or non-tangible computer readable transmission media that may also participate in the provision of software, application programs, instructions and/or data to the computing device 3200 include radio or infra-red transmission channels as well as a network connection to another computer or networked device, and the Internet or Intranets including e-mail transmissions and information recorded on Websites and the like.

The computer programs (also called computer program code) are stored in main memory 3208 and/or secondary memory 3210. Computer programs can also be received via the communication interface 3224. Such computer programs, when executed, enable the computing device 3200 to perform one or more features of embodiments discussed herein. In various embodiments, the computer programs, when executed, enable the processor 3204 to perform features of the above-described embodiments. Accordingly, such computer programs represent controllers of the computer system 3200.

Software may be stored in a computer program product and loaded into the computing device 3200 using the removable storage drive 3214, the storage drive 3212, or the interface 3220. The computer program product may be a non-transitory computer readable medium. Alternatively, the computer program product may be downloaded to the computer system 3200 over the communications path 3226. The software, when executed by the processor 3204, causes the computing device 3200 to perform the necessary operations to execute the method as shown in FIG. 3.

It is to be understood that the embodiment of FIG. 32 is presented merely by way of example to explain the operation and structure of the apparatus 400. Therefore, in some embodiments one or more features of the computing device 3200 may be omitted. Also, in some embodiments, one or more features of the computing device 3200 may be combined together. Additionally, in some embodiments, one or more features of the computing device 3200 may be split into one or more component parts.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

What is claimed is:

1. A method for identifying a posture condition of a person comprising:
detecting a rotation of one or more body parts of the person around an upright center axis of the person; and
calculating an asymmetric score of the one or more body parts based on the rotation of the one or more body parts of the person, the asymmetric score relating to a level of the posture condition of the person.

2. The method of claim 1, further comprising:
detecting an angle of a first line connecting two body part positions of the person from an image of the person against a reference line, wherein the detection of the rotation of the one or more body parts of the person around the upright center axis of the person is based on the angle.

3. The method of claim 2, wherein the first line connects a left side body part position and a right side body part position of a first body part of the person across a frontal plane and the reference line comprises a second line connecting a left side body part position and a right side body part position of a second body part of the person across the frontal plane.

4. The method of claim 2, wherein the reference line is a third line connecting another two body part positions of the person.

5. The method of claim 1, further comprising:
detecting a first distance of a body part position from a nearest point along the upright center axis of the person; wherein the detection of the rotation of the one or more body parts of the person and/or the calculation of the asymmetric score of the one or more body parts of the person is based on the first distance.

6. The method of claim 1, further comprising:
Detecting a second distance of a body part position from one of (i) two body part positions of the person, (ii) a middle point between the two body part positions or (iii) a fourth line connecting the two body part positions, wherein the detection of the rotation of the one or more body parts of the person and/or the calculation of the asymmetric score of the one or more body parts of the person is based on the second distance.

7. The method of claim 1, further comprising:
detecting a plurality of body part positions of the person based on relative positions of a plurality of body parts in one or more images in which the person is detected, wherein each of plurality of body part positions corresponds to a body part of the person.

8. The method of claim 7, wherein detecting the plurality of body part positions of the person comprises:
estimating a body part position of the person based on one of the plurality of body part positions, wherein the plurality of body part positions of the person further comprises the estimated body part position.

9. The method of claim 1, further comprising:
receiving demographic data relating to the person; wherein the calculation of the asymmetric score is further based on the demographic data.

10. The method of claim 1, wherein the asymmetric score is one of a plurality of asymmetric scores relating to a plurality of body parts, the method further comprising:
calculating the level of the posture condition of the person based on the plurality of asymmetric scores.

11. The method of claim 9, further comprising:
comparing each of the plurality of asymmetric scores with other asymmetric scores of the plurality of asymmetric scores;
identifying one or more asymmetric scores having a higher score among the plurality of asymmetric scores; and
identifying a set of posture correction programs based on a result of the identification.

12. An apparatus for identifying a posture condition of a person comprising, the apparatus comprising:
at least one processor; and
at least one memory including computer program code;
the at least one memory and the computer program code configured to, with at least one processor, cause the server at least to:
detect a rotation of one or more body parts of the person around an upright center axis of the person; and
calculate an asymmetric score of the one or more body parts based on the rotation of the one or more body parts of the person, the asymmetric score relating to a level of the posture condition of the person.

13. The apparatus of claim 12, wherein the at least one memory and the computer program code configured to, with at least one processor, cause the server at least to further:
detect an angle of a first line connecting two body part positions of the person from an image of the person against a reference line; and
detect the rotation of the one or more body parts of the person around the upright center axis of the person based on the angle.

14. The apparatus of claim 13, wherein the first line connects a left side body part position and a right side body part position of a first body part of the person across a frontal plane and the reference line comprises a second line connecting a left side body part position and a right side body part position of a second body part of the person across the frontal plane.

15. The apparatus of claim 13, wherein the reference line is a third line connecting another two body part positions of the person.

16. The apparatus of claim 12, wherein the at least one memory and the computer program code configured to, with at least one processor, cause the server at least to further:
detect a first distance of a body part position from a nearest point along the upright center axis of the person; and
detect the rotation of the one or more body parts of the person and/or calculate the asymmetric score of the one or more body parts of the person based on the first distance.

17. The apparatus of claim 12, wherein the at least one memory and the computer program code configured to, with at least one processor, cause the server at least to further:
detect a second distance of a body part position from one of (i) two body part positions of the person, (ii) a middle point between the two body part positions or (iii) a fourth line connecting the two body part positions; and
detect the rotation of the one or more body parts of the person and/or calculate the asymmetric score of the one or more body parts of the person based on the second distance.

18. The apparatus of claim 12, wherein the at least one memory and the computer program code configured to, with at least one processor, cause the server at least to further:
detect a plurality of body part positions of the person based on relative positions of a plurality of body parts in one or more images in which the person is detected, wherein each of plurality of body part positions corresponds to a body part of the person.

19. The apparatus of claim 18, wherein the at least one memory and the computer program code configured to, with at least one processor, cause the server at least to further:
estimate a body part position of the person based on one of the plurality of body part positions, wherein the plurality of body part positions of the person further comprises the estimated body part position.

20. The apparatus of claim 12, wherein the at least one memory and the computer program code configured to, with at least one processor, cause the server at least to further:
receive demographic data relating to the person; and
calculate the asymmetric score further based on the demographic data.

21. The apparatus of claim 12, wherein the at least one memory and the computer program code configured to, with at least one processor, cause the server at least to further:
calculate the level of the posture condition of the person based on the plurality of asymmetric scores.

22. The apparatus of claim 21, wherein the at least one memory and the computer program code configured to, with at least one processor, cause the server at least to further:
compare each of the plurality of asymmetric scores with other asymmetric scores of the plurality of asymmetric scores;
identify one or more asymmetric scores having a higher score among the plurality of asymmetric scores; and
identify a set of posture correction programs based on a result of the identification.

23. A system for identifying a posture condition of a person comprising the apparatus of claim 12 and one or more image capturing apparatuses configured to capture one or more images of the person, wherein the one or more images comprises an image of the person across a frontal plane and/or an image of the person across a sagittal plane.
